(19) United States
(12) Patent Application Publication
(10) Pub. No.: US 2007/0093660 A1
(43) Pub. Date: Apr. 26, 2007

(54) Method for the production of statins

(76) Inventors: Vitali Tararov, Moskau (RU); Gerd Konig, Zwickau (DE); Armin Borner, Rostock (DE)

Correspondence Address: SMITH PATENT CONSULTING CONSULTING, LLC, 3309 DUKE STREET, ALEXANDRIA, VA, 22314, US

(21) Appl. No.: 10/578,706

(22) PCT Filed: Nov. 9, 2004
(86) PCT No.: PCT/EP04/12659

§ 371 Date: May 10, 2006

(30) Foreign Application Priority Data
Nov. 11, 2003 (DE) 103 52 659.5

(51) Int. Cl. C07D 405/02; C07D 239/02

(52) U.S. Cl. 544/330; 548/465; 546/281.7; 549/293

(57) ABSTRACT

According to the invention, a process for the preparation of a statin is provided, comprising the following steps:
a) preparation of a compound of the formula II 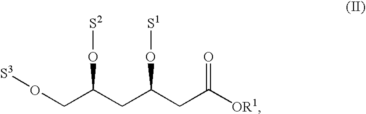 in which
S¹ denotes a hydrogen atom or a hydroxylprotective group,
S² and S³, independently of one another, denote hydroxylprotective groups and
R¹ represents a hydrogen atom or a carboxylprotective group,
   by stereoselective hydrogenation of a compound of the formula III 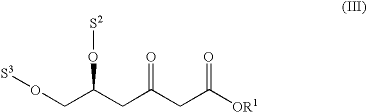
   to give a compound of the formula II-a 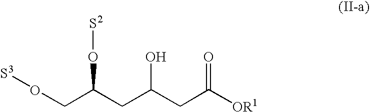 and optionally introduction of a hydroxylprotective group and
   b) lactonization of the compound of the formula II to give a compound of the formula I-a

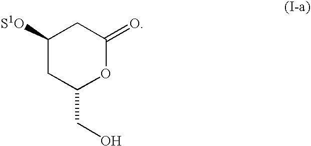

The present invention relates to a process for the preparation of statins, which are known to be HMG-CoA reductase inhibitors. Some of the intermediate compounds for use in the process according to the invention are novel compounds, and the invention also relates to these novel intermediate compounds.

Statins are a known class of active substances which inhibit the enzyme hydroxymethylglutaryl(HMG)-CoA reductase. The active substances are widely used, in particular as cholesterol depressants in the blood. Known statins are, for example, cerivastatin, fluvastatin, itavastatin, BMY22089, rosuvastatin, glenvastatin and atorvastatin. Synthesis routes for the statins are known and are described in a large number of publications. Statins are in principle based on an aromatic, heterocyclic or aromatic-heterocyclic, substituted or unsubstituted, mono-, di- or polycyclic ring system to which the so-called statin side chain is attached either in open-chain form or in the lactone form 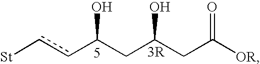
where St represents the ring system described above, i.e. the radical of the statin. As used in the context of this description, the term “statin” is also understood as meaning the pharmaceutically tolerated salts, hydrates, solvates, esters and ethers of the statins described in the prior art.

Of decisive importance for the efficacy of the statins is the three-dimensional arrangement of the hydroxyl groups in the statin side chain, as shown in the above formula. In the synthesis of the statin, it is economically expedient to establish the stereochemistry in a very early step and to carry out the further steps with retention of the stereochemistry, i.e. stereoselectively, in order to obtain as high yields as possible of the end product (products having a different stereochemistry must be separated off).

Processes for the preparation of statins have long been known and are the subject of chemical research. An early publication of 1984 (J. Org. Chem. Vol. 49, No. 21, 1984, 3994-4003) describes, inter alia, the following reaction 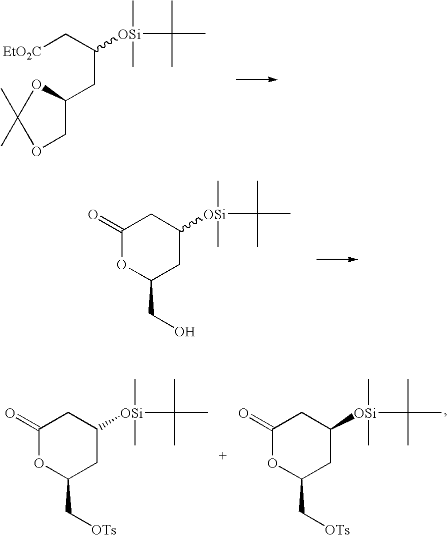
in which Ts represents a tosyl protective group. The compound 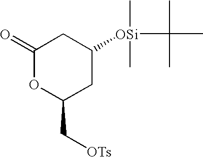
is regarded as a possible intermediate for the preparation of the lactone radical of compactin, one of the first statins, but the publication assumes that cleavage of the racemate 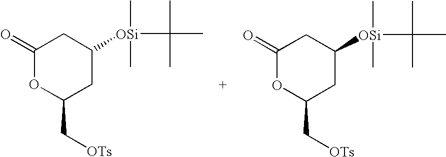
is required for isolating this compound which makes the overall process ineffective.

For the preparation of the modern statins, too, the prior art then adopted other routes which did not involve this silylated intermediate compound. The use of an alcohol whose hydroxyl groups in the 5- and 6-position are protected by a bridging protective group, like the starting compound of the above synthesis, was also employed only in isolated cases with moderate success in statin synthesis, for example in EP-A 374 922, which discloses the preparation of the compound ethyl 5,6-O-isopropylidene-3,5,6-trihydroxyhexanoate. The end product of this synthesis contains the desired (3R,5S)-isomer, but only in a ratio of 78:22, which is unsatisfactory for commercial purposes. A conversion of this compound into a lactone did not take place.

In comparison, more recent methods for improving the statin synthesis, as described, for example, in EP-A 583 171 take place via intermediate compounds in which the hydroxyl protective groups in the 3- and 5-position of the trihydroxyhexanoate are protected by a bridging protective group, or via intermediate compounds in which bridging protective groups are completely dispensed with, which methods do not take place via a lactonization reaction.

Typical examples of the direction in which the prior art is moving in the synthesis of statins are described, for example, in WO 03/004450 and WO 03/004456. These publications disclose so-called “key intermediates” 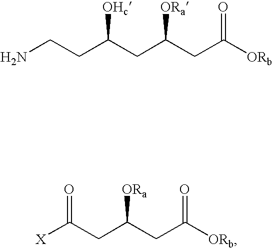
which can be coupled with the radical of the statin after further reactions. These “key intermediates” are prepared by hydrolysing a racemic mixture of the compound 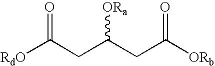
by an enantioselective catalyst.

This process has the advantage that the stereochemistry of the statin side chain is established in an early stage, but the procedure is very complex, the stereoselective hydrolysis of the compound 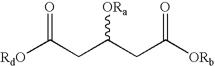
is associated with a lower yield, and there is the risk that the stereochemistry of the side chain will be lost in the further procedure.

Processes for the preparation of HMG-CoA reductase inhibitors or the preparation of the statin side chain are likewise described in Prasad, K. et al., Tetrahedron: Asymmetry Vol. 1, No. 5, pages 307-310, 1990; Prasad, K. et al., Tetrahedron Letters, Vol. 25, No. 32, pages 3391-3394, 1984; Bennett, F. et al., J. Chem. SOC. Perkins Trans. 1, pages 133-140, 1991; DE 35 30 798 and EP-A 1 288 213.

There is a considerable need for further processes for the preparation of statins, which are economical and with the aid of which the statins can be prepared in a simple manner, in high yield and with the use of fewer process stages.

According to the invention, it was found that a statin synthesis which is based on the early experiments, as described, for example, in the publication J. Org. Chem., Vol. 49, No. 21, 1984, 3994-4003, but was regarded there as being not very promising, gives the statins with the desired statin side chain in good yield and high optical purity. Some of the intermediates are known from the literature, while other intermediates are novel compounds. According to the invention, a process by means of which these intermediates can be prepared in a simple manner and in higher yield was also found.

The invention therefore relates to a process for the preparation of a statin, comprising the following steps:
a) preparation of a compound of the formula II 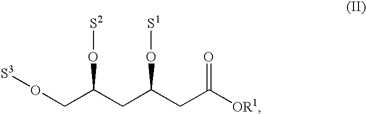
   in which
S¹ denotes a hydrogen atom or a hydroxylprotective group,
S² and S³ independently of one another, denote hydroxylprotective groups or S² and S³ together denote a bridging hydroxylprotective group and
R¹ represents a hydrogen atom or a carboxylprotective group,
   by stereoselective hydrogenation of a compound of the formula III 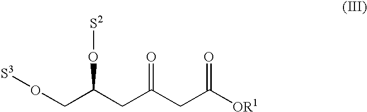
   to give a compound of the formula II-a 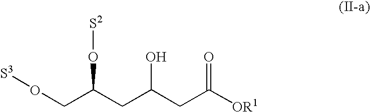
   and optionally introduction of the hydroxylprotective group and
b) Lactonization of the compound of the formula II to give a compound of the formula

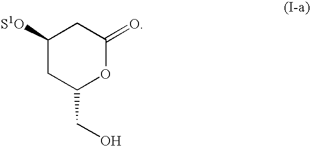

The invention also relates to a process for the preparation of an intermediate of the formula (I-a) 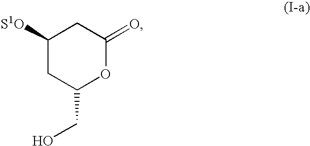
as defined above.

The radical S¹ represents a hydrogen atom or a hydroxylprotective group. Hydroxylprotective groups are known in the prior art, and reference may be made, for example, to the general literature reference Protective Groups in Organic Synthesis, Theodora W. Greene and Peter G. M. Wuts, 2nd edition, John Wiley & Sons. Suitable hydroxylprotective groups are also mentioned, for example, in WO 03/004450, which is hereby incorporated by reference. According to the invention, hydroxylprotective groups having 4 to 10 carbon atoms and optionally 1 to 3 heteroatoms are preferred. Particularly preferably, the hydroxylprotective group contains a silicon atom, 5 to 10 carbon atoms and no further heteroatoms. The hydroxyl protective group S¹ is particularly preferably a trimethylsilyl, triisopropylsilyl, triethylsilyl, tert-butyldimethylsilyl, di-tert-butylmethylsilyl, tert-butyldiphenylsilyl, triphenylsilyl, diphenylmethylsilyl or tris(trimethylsilyl) protective group. Most preferably, the hydroxyl protective group S¹ is a tert-butyldimethylsilyl group. Protective groups of the general formulae R—O—C(O)— and R—C(O)—, where R represents an alkyl group, in particular a C₁-C₆-alkyl group, such as a tert-butyl group, or an aryl group, in particular a C₅-C₁₀-aryl group, such as a phenyl group, or an alkylaryl group, in particular a C₁-C₆-alkyl-C₅-C₁₀-aryl group, are also preferred.

The radicals S² and S³ may be customary hydroxylprotective groups, and it is possible to use the same hydroxylprotective groups which were mentioned above in relation to the hydroxyl protective group S¹. Once again, reference may be made to the standard work Protective Groups in Organic Synthesis, Theodora W. Greene and Peter G. M. Wuts, 2nd edition, John Wiley & Sons. However, S² and S³ preferably together form a bridging hydroxylprotective group, as known in principle. Examples of suitable bridging hydroxylprotective groups are disclosed in WO 03/004450, which is hereby incorporated by reference. Particularly preferably, the protective groups S² and S³ together form an isopropylidene protective group.

The radical R¹ denotes a hydrogen atom or a carboxylprotective group. Carboxylprotective groups are known to the person skilled in the art and are described, for example, in Protective Groups in Organic Synthesis, Theodora W. Greene and Peter G. M. Wuts, 2nd edition, John Wiley & Sons. R¹ may denote, for example, a hydrogen atom, or a C₁₋₃-alkyl or C₅₋₁₀-aryl radical, which are optionally substituted by one or more radicals which, independently of one another, are selected from halogen atoms, C₁-C₁₀-alkyl radicals, C₅-C₁₀-alkoxy radicals, heterocycles which are composed of 0 to 10 carbon atoms, preferably 1 to 5 carbon atoms, and 1 to 10 heteroatoms, preferably 1 to 5 heteroatoms, selected from sulphur, nitrogen and oxygen atoms, and functional groups. R¹ preferably denotes a C₁₋₈-alkyl or C₅₋₁₀-aryl radical, which are optionally substituted by one or more radicals which, independently of one another, are selected from halogen atoms, tetrazolyl, C₁₋₈-alkyl, C₁₋₈-alkoxy, nitro and cyano groups.

The radical R¹ is particularly preferably a C₁₋₈-alkyl radical, in particular C₁₋₃-alkyl radical, an ethyl radical being most preferred, in particular if the radical S¹ represents a tert-butyidimethylsilyl group.

The compound of the formula I-a 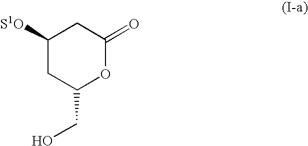
can be converted in a simple manner into compounds of the formula I 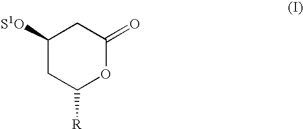
which are important intermediate compounds in the preparation of statins.

In the compounds of the formula I 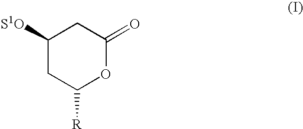
the radical S¹ is as defined above. The radical R is a radical via which the compound of the formula I can be coupled to the radical of the statin, in particular a —CH₂R², —CHO, —CH═P(R³)₃, 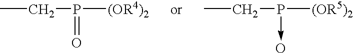
-group. The group —CH═P(R³)₃ is present in equilibrium with the group —CH—⁺P(R³)₃. These therefore also include groups —CH₂—P⁺(R³)₃M⁻, where M⁻ represents a customary opposite ion, e.g. Hal⁻(Hal=Cl, Br or I) or ⁻O-Tos.

If the radical R is a —CH═P(R³)₃, 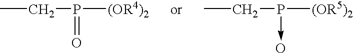
group, the compound of the formula I is a Wittig reagent or a Horner-Wittig reagent which can subsequently undergo a Wittig reaction or a Horner-Wittig reaction with the appropriately functionalised ring system St of the statin. The ring system St with which the compound of the formula I is reacted to give the statin should in this case preferably carry an aldehyde group at the coupling site.

The radicals R³, R⁴ and R⁵ are preferably the customary groups which complete a Wittig radical or a Horner-Wittig radical, so that the compounds can undergo a Wittig reaction or a Horner-Wittig reaction. The radical R³ therefore usually denotes a C₅- to C₁₀-aryl radical which is optionally substituted by one or two C₁-C₄-alkyl radicals and/or halogen atoms, a C₁-C₄-alkyl radical or a C₅-C₁₀-cycloalkyl radical, in particular a phenyl radical, a n-C₁-C₄-alkyl radical or a cyclohexyl radical. The phenyl radical is preferably unsubstituted. Preferably, the phenyl radical is also substituted by one or two n-C₁-C₄-alkyl radicals or chlorine atoms. The radical R⁴ is preferably a C₁-C₄-alkyl radical, in particular a n-C₁-C₄-alkyl radical, particularly preferably an ethyl group, and the radical R⁵ is preferably a C₅-C₁₀-aryl radical or a C₁-C₆-alkyl radical, in particular a C₅-C₁₀-aryl radical or a C₁-C₄-alkyl group, particularly preferably a phenyl, methyl or ethyl group. However, these radicals are not particularly limited, provided it is possible to carry out the subsequently required Wittig (or Horner-Wittig) reaction with them.

If the radical R in the compound of the formula I represents an aldehyde group, the ring system St with which the compound of the formula I is reacted to give the corresponding statin should have an appropriate functional group so that a Wittig reaction or a Horner-Wittig reaction can be carried out.

The Wittig reaction and the Horner-Wittig reaction are known reactions, and reference may be made to relevant textbooks of organic chemistry, for example to March, Advanced Organic Chemistry, 4th edition, 1992, John Wiley and Sons.

If the radical R represents a radical —CH₂R², the radical R² represents, according to the invention, a halogen atom, in particular a chlorine, bromine or iodine atom, a cyanide group (—C≡N), a —CH₂NH₂— group or a group SO₂—R⁶ or a leaving group.

If R² represents a cyanide group, the compound of the formula I is in particular an intermediate for the preparation of a compound of the formula I in which R² represents a —CH₂NH₂ group. The compound of the formula I in which R² represents a —CH₂NH₂ group is a particularly preferred intermediate which is suitable for the preparation of atorvastatin.

A compound of the formula I in which the radical R² represents a cyanide group can be converted by hydrogenation into a compound of the formula I in which R² represents a —CH₂NH₂ group.

The compounds of the formula I in which the radical R² denotes a radical —SO₂R⁶ can, depending on the compounds in which the radical R denotes a Wittig radical or a Horner-Wittig radical, be reacted with a ring system St, which carries, for example, an aldehyde group as a coupling group, to give a statin. The corresponding sulphones can be obtained either directly from the alcohols of the formula I-a or from the tosylates of the formula I, for example by reaction with sulphides and subsequent oxidation with peroxides or H₂O₂, as described, for example, in Tetrahedron Letters, 1973, 49, 4833-4836; Synlett 1988, 26-28 or J. Am. Chem. Soc. 2001, 123, 10772-10773.

The radical R⁶ denotes a hydrogen atom or a C₁₋₃-alkyl or C₅₋₁₀-aryl radical which are optionally substituted or one or more radicals which, independently of one another, are selected from halogen atoms, C₁-C₁₀-alkyl radicals, C₁-C₁₀-alkoxy radicals, heterocycles which are composed of 0 to 10 carbon atoms, preferably 1 to 5 carbon atoms, and 1 to 10 heteroatoms, preferably 1 to 5 heteroatoms, selected from sulphur, nitrogen and oxygen atoms, and functional groups. The radical R⁶ preferably denotes a hydrogen atom or a C₁₋₈-alkyl or C₅₋₁₀-aryl radical which are optionally substituted by one or more radicals which, independently of one another, are selected from halogen atoms, tetrazolyl, C₁₋₈-alkyl, C₁₋₈-alkoxy, nitro and cyano groups.

According to the invention, the radical R² may also be a customary leaving group which, in a nucleophilic substitution reaction, permits coupling with a suitably substituted statin radical. Suitable leaving groups are known in organic chemistry, and reference can preferably be made here to halogen atoms, in particular chlorine, bromine and iodine atoms, and radicals —O—SO₂—R where R represents an alkyl, aryl or alkylaryl radical, preferably having not more than 20 carbon atoms, particularly preferably a C₁-C₆-alkyl radical or a C₅-C₁₀-aryl radical, which is optionally substituted by one or two C₁-C₆-alkyl radicals, such as a phenyl group or a p-tolyl group. The radical —O—SO₂—R is preferably a —O-Tos group, where Tos represents a tosyl group.

The radical R² is particularly preferably a cyanide group, a —CH₂NH₂ group or a radical SO₂R⁶. For all meanings of R² the hydroxylprotective group S¹ is as defined above, in particular also with respect to its preferred meanings.

The compounds of the formula I in which the radical R² denotes a halogen atom can preferably be prepared directly from the compounds of the formula I-a. Compounds of the formula I in which the radical R² denotes a halogen atom can also be prepared from compounds of the formula I in which the radical R² denotes another leaving group, in particular a O-tosyl group. The preparation of compounds of the formula I in which the radical R² represents a —O-tosyl group from the compounds of the formula I-a is disclosed in the prior art. The compounds of the formula I in which the radical R² denotes a halogen atom can be converted, for example by reaction with a compound P(R⁴)₃, into a compound of the formula I in which the radical R denotes a —CH═P(R³)₃ group.

The compounds of the formula I-a can be reacted according to the following scheme to give preferred compounds of the formula I: 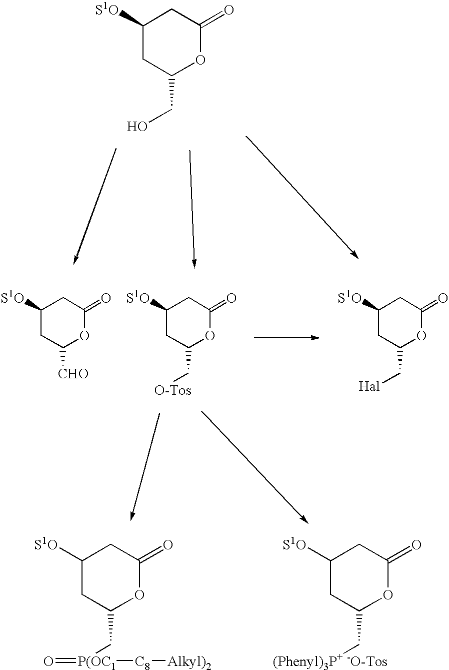
where Hal denotes a halogen atom.

The oxidation of a primary OH group to an aldehyde group can be effected, for example, by means of a Swern oxidation or by oxidation with Cr(VI): (PyH)₂Cr₂O₇—Handbook of Reagents for Organic Synthesis “Oxidizing and Reducing Agents”, Ed. S. D. Burke, R. L. Danheiser, John Wiley & Sons Ltd. 1999, pages 330-334 or with Cr(VI): HPyCrClO₃—Handbook of Reagents for Organic Synthesis “Oxidizing and Reducing Agents”, Ed. S. D. Burke, R. L. Danheiser, John Wiley & Sons Ltd. 1999, pages 323-330.

The conversion of the tosyl groups into a halide can be effected, for example, as described in Weygand/Hilgetag, 4^{th} edition, 1970, pages 232-235. The conversion of the tosyl groups into cyanide is described, for example, in Organikum, 16th edition, 1986, 211-216; P. Kurtz in: Houben-Weil, vol. 8, 1952, pages 290-311; D. T. Mowry, Chem. Rev. 1948, 42, 189-284.

Details of the preparation of the above compounds can also be obtained, for example, from the following publications:
Journal of the Chemical Society, Perkin Transactions 1: Organic and Bioorganic Chemistry (1972-1999) (1988), (8), 2291-5; (for the aldehydes)
Journal of Organic Chemistry (2001), 66 (20), 6803-6806; (for the tosylate)
Tetrahedron (1995), 51 (48), 13217-38; (for the tosylate)
Journal of the Chemical Society, Perkin Transactions 1: Organic and Bioorganic Chemistry (1995), (13), 1641-3; (for the tosylate)
Journal of Organic Chemistry (1984), 49 (21), 3994-4003; (for the tosylate)
Fujisawa, Kamotsu et al., JP 10087568 A2, 1998, 0407 Heisei; (for the chloride)
Chemistry Letters (1997), (8), 765-766; (for the chloride)
Tetrahedron Letters (1996), 37 (33), 6001-6004; (for the iodide)
Journal of the Chemical Society, Perkin Transactions 1: Organic and Bioorganic Chemistry (1972-1999) (1991), (1), 13340 (for the iodide) and
Tetrahedron Letters (1988), 29 (38), 4865-8 (for the iodide).

Most compounds of the formula I are novel, and the present invention also relates to these novel compounds. Compounds of the formula I in which the radical S¹ represents a tert-butyldimethylsilyl group and the radical R represents a —CHO, —CH₂—O-Tos, —CH₂Cl or —CH₂I group are known and are therefore not claimed as a compound. Particularly preferred novel compounds according to the invention are compounds of the formula I, where the radical S¹ represents a tert-butyldimethylsilyl group and the radical R represents a —CH₂R², —CH═P(R³)₃, —CH₂—P⁺(R³)₃M⁻, 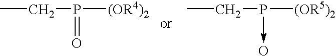
group, where R² represents a bromine atom, C≡N, a —CH₂NH₂ group or a radical —SO₂—R⁶, and R³, R⁴, R⁵, R⁶ and M⁻ are as defined above. These compounds can be easily prepared on the basis of the above statements.

WO 93/06235 discloses, in general form, certain tetrahydropyran-2-one isomers.

Preferred compounds according to the invention of the formula I 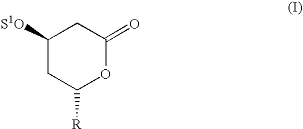
are those compounds
in which
S¹ is as defined above,
R denotes —CH₂R², —CHO or —CH═P(R³)₃,
R² denotes —CH₂NH₂, —SO₂—R⁶ or a radical —O—SO₂R⁷, where R⁷ represents an alkyl, aryl or alkylaryl radical,
   with the proviso that the radical S¹ does not represent a tert-butyldimethylsilyl group if the radical R represents a CHO group or CH₂—OTos group,
R³ completes a Wittig radical or a Horner-Wittig radical,
R⁶ denotes a hydrogen atom or a C₁₋₃-alkyl- or C₅₋₁₀-aryl radical, which are optionally substituted by one or more radicals which, independently of one another, are selected from halogen atoms, heterocycles which contain 0 to 10 carbon atoms and 1 to 10 heteroatoms selected from sulphur, nitrogen and oxygen atoms, and functional groups and
M⁻ represents an opposite ion.

Even more preferred are those compounds in which the radical S¹ represents the tert-butyldimethylsilyl group and the radical R represents a —CH₂R² or —CH═P(R³)₃ group, where R² represents a —CH₂NH₂ group or a radical —SO₂—R⁶,
R³ completes a Wittig radical or a Horner-Wittig radical,
R⁶ denotes a hydrogen atom or a C₁₋₃-alkyl- or C₅₋₁₀-aryl radical, which are optionally substituted by one or more radicals which, independently of one another, are selected from halogen atoms, heterocycles which contain 0 to 10 carbon atoms and 1 to 10 heteroatoms selected from sulphur, nitrogen and oxygen atoms, and functional groups and
M⁻ represents an opposite ion.

Reference is also made to the following examples.

According to the invention, the compounds of the formula I-a 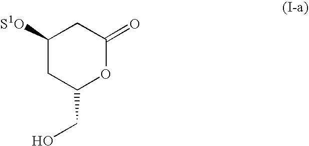
are prepared starting from compound II 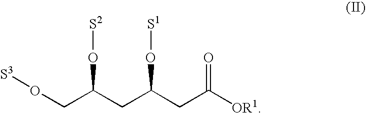
where the radicals S¹, S², S³ and R¹ are as defined above.

This process step is in principle known from the literature and can be effected, for example, by heating in acetic acid. For example, reference is made to the following publications:
Journal of Organic Chemistry (1984), 49 (21), 3994-4003;
EP-A 1 234 885;
U.S. Pat. No. 6,417,374;
Journal of Organic Chemistry (2001), 66 (20), 6803-6806;
Tetrahedron (1995), 51 (48), 13217-38;
Journal of the Chemical Society, Perkin Transactions 1: Organic and Bioorganic Chemistry (1995), (13), 1641-3,
   and which are hereby fully incorporated by reference.

The compound of the formula II 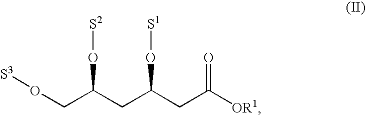
in which S¹ represents a hydroxyl group can easily be prepared from compounds of the formula II, in which the radical S¹ represents a hydrogen atom, i.e. compounds of the formula II-a, for example by reaction with a compound S¹-A, where A represents a customary leaving group, such as a halogen atom (e.g. chlorine, bromine or iodine), and S¹ represents the hydroxyl protective group.

The particularly preferred compound of the formula II, in which the protective group S¹ represents a tert-butyldimethylsilyl protective group, can advantageously be prepared, for example, by reacting the compound II-a with ClSiMe₂tert-butyl in a mixture of DMF and imidazole.

The compound of the formula II-a 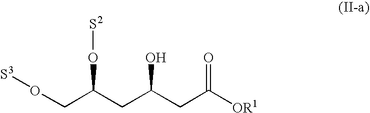
can be prepared with high stereoselectivity from the compound of the formula III 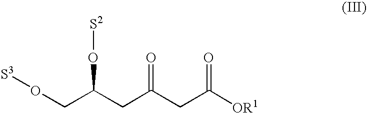
for example by hydrogenation with hydrogen at room temperature (25° C.) under elevated pressure in the range of from 20 to 80 bar, in particular from 30 to 70 bar, e.g. about 50 bar, in a suitable solvent, preferably a polar protic solvent, in particular in a C₁- to C₆-alcohol, such as methanol or ethanol. The hydrogenation is preferably effected using a so-called Ru-BINAP catalyst, as described, for example in Tetrahedron Lett. 1991, 32, 4163, and in WO 95/18784, and these two publications are hereby fully incorporated by reference for the definition and the preparation of the preferred catalyst for carrying out the process according to the invention.

The catalysts most preferred according to the invention have the structure

(R)—Ru(BINAP)Cl₂×NEt₃ (Et=Ethyl) or

(R)—Ru(BINAP)Cl₂×DMF×NEt₃,

where BINAP has the formula 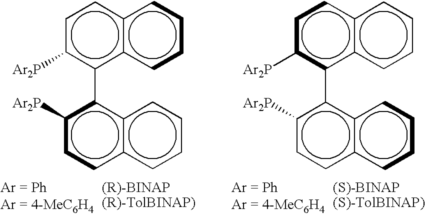
(Ph=phenyl), and can be prepared as described, for example, in Tetrahedron Lett. 1991, 32, 4163. Instead of (R)-BINAP, (R)-Tol BINAP can also advantageously be used. With the catalyst (R)—Ru(BINAP)Cl₂.NEt₃, a syn/anti ratio of ≦80, in particular of ≦90, preferably ≦95, more preferably ≦99, can be achieved in the reaction according to the invention, which corresponds to a molar ratio of the desired (3R,5S)-isomer to the undesired isomer of 80:20 or better. On the other hand, only a molar ratio of 78:22 could be achieved in the prior art, as, described, for example, in EP-A 374 922. Even with the corresponding (S)-BINAP catalyst, a stereoselective reaction is obtained, but the unfavourable anti-isomer predominates. The invention therefore also relates to a process for the stereoselective hydrogenation of a compound of the formula III to give a compound of the formula II-a, in particular with the use of a (R)—RuBINAP or (R)—RuTolBINAP catalyst, these catalysts being defined as above and being described, for example, in WO 95/18784 or in Tetrahedron Lett. 1991, 32, 4163, and the hydrogenation giving a molar ratio of the compound of the formula II-a 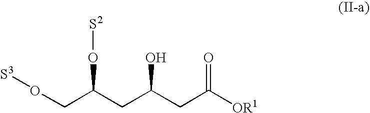
to the corresponding anti-compound 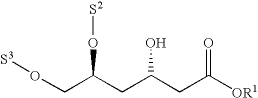
of 80:20 or more, in particular of 90:10 or more, in particular of 95:5 or more and most preferably of 99:1 or more. As a rule it is therefore no longer necessary in the process according to the invention to separate off the undesired anti-isomer. If this should nevertheless be necessary, it can be effected in a known manner.

The compound of the formula III is easily obtainable, for example according to the process of Angew. Chem. 1979, 91, 76-77, from the compound of the formula IV 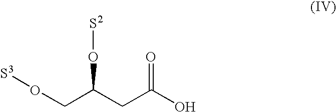
which can be obtained, for example, in a known manner from the commercially available and economical S-malic acid.

The conversion of the compound IV into the compound III is advantageously effected by first activating the carboxyl group of the compound IV with a suitable activating agent, such as N,N′-carbonyldiimidazole. The activated compound is then reacted with a compound M¹R⁷₂X₀₋₁. Here, M¹ is a divalent or trivalent metal cation, in particular a metal cation of the second or third main group or the second or third subgroup of the Periodic Table of the Elements, in particular a magnesium, calcium, zinc or aluminium ion, particularly preferably a magnesium, zinc or aluminium ion (Mg²⁺, Zn²⁺ or Al³⁺ ions), and the radical R⁷ is a suitable carboxylic acid radical, in particular a partially esterified dicarboxylic acid radical, such as, for example, a C₁₋₄—O₂C(CH₂)₁₋₄CO₂ radical, e.g. an EtO₂CCH₂CO₂ radical. A further example of a suitable radical R⁷ is a C₁₋₆COO radical, such as a CH₃COO radical. The two radicals R⁷ on the metal cation may be identical, but two different radicals R⁷ may also be present on the metal ion. The radical X represents an optionally present monovalent opposite ion which serves for charge compensation if the metal cation M¹ is a trivalent ion. Particularly preferably, the two radicals R⁷ are different; for example, one of the radicals R⁷ is a C₁₋₄—O₂C(CH₂)₁₋₄CO₂ radical and the other radical is a C₁-C₆COO radical. Very good results can be obtained, for example, with the compounds Mg(CH₃COO⁻)(EtO₂CCH₂CO₂⁻), Zn(EtO₂CCH₂CO₂⁻)(EtO₂CCH₂COO⁻) or AlCl(EtO₂CCH₂CO₂⁻)(EtO₂CCH₂COO⁻). Other combinations of the above radicals are of course also possible according to the invention. The reaction can be effected, for example, at room temperature in THF and it was possible to achieve yields of more than 60%, preferably of more than 70%.

The metal salts can preferably be prepared in situ, for example by reacting the corresponding metal powder with the corresponding acid (e.g. EtO₂CCH₂COOH) with reflux in a suitable solvent, such as an ether, e.g. THF.

By means of the process according to the invention, all statins which have the side chain 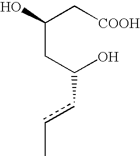
where the dashed line represents an optionally present bond, or the corresponding lactone can preferably be prepared.

The following statins may preferably be mentioned: 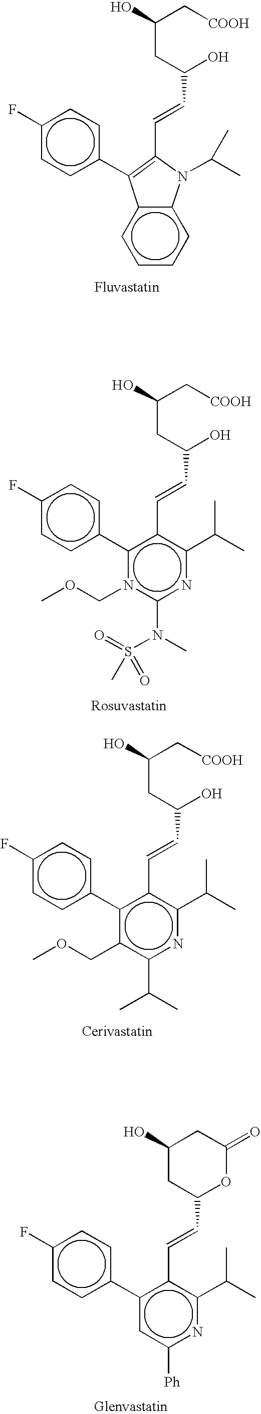 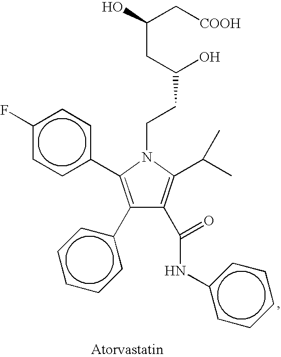
but also, for example, itavastatin and BMY22089.

Statins which have a side group 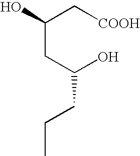
can be obtained by hydrogenation of the corresponding statins which have the side group

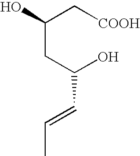

The hydrogenation preferably takes place on a precursor of the statin, in which the hydroxyl group is protected, i.e. on a statin having the side chain 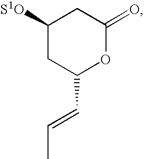
where S¹ represents a hydroxylprotective group and is defined as above.

The elimination of the protective group S¹ (if present) and the opening of the lactone ring preferably takes place by hydrolysis as the last step of the statin synthesis according to the invention.

According to the invention, the coupling of the compound of the formula I with the ring system St which represents the radical of the statin preferably takes place by a Wittig reaction or by a Horner-Wittig reaction. Here, either the radical St of the statin is functionalized with an aldehyde group, in particular if the compound of the formula I carries a Wittig or Horner-Wittig functionality, or the ring system of the radical St is provided with a Wittig or Horner-Wittig functionality if the compound of the formula I carries an aldehyde group. Processes for the preparation of correspondingly functionalized ring systems St are described, for example, in the publications WO 84/02131, EP-A 244 364 and EP 521 471, which are hereby fully incorporated by reference. Functionalized ring systems St, which are not expressly mentioned in these publications can be prepared in a corresponding manner. Here, reference may be made, for example, to WO 01/04100 and WO 03/006439.

A general process scheme for the preparation of the statins is as follows 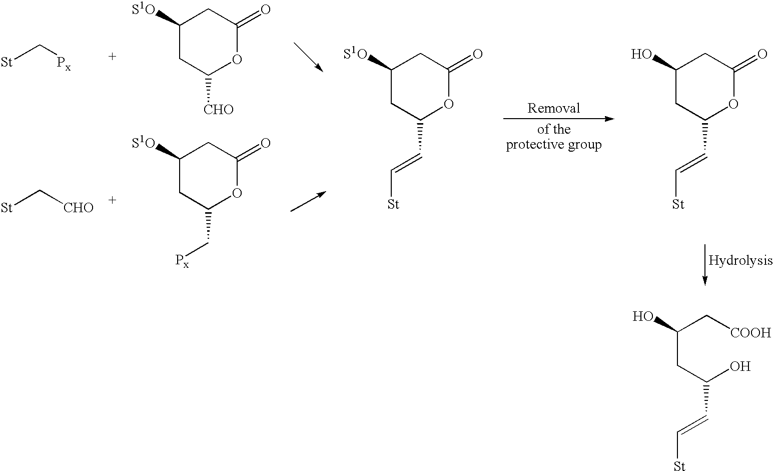
where S¹ is as defined above and Pₓ represents a Wittig group or a Horner-Wittig group as defined above, in particular a —P⁺(Phenyl)₃⁻OTos group and St represents the radical of the statin, e.g. 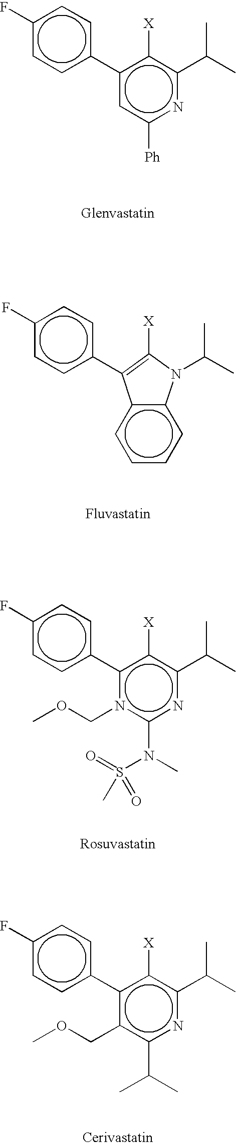
(X indicates a bonding site in each case)

In the above scheme, the radical Pₓ may also represent a radical —S(O)₂—R⁶, where R⁶ is as defined above.

An exemplary synthesis scheme for the preparation of atorvastatin is as follows

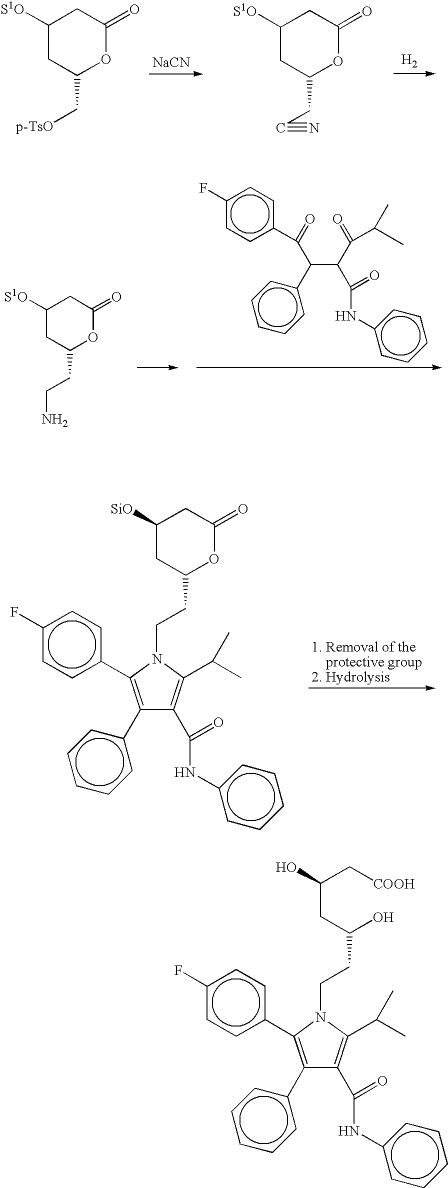

The diketone 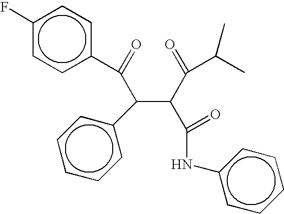
can be prepared, for example, as described in WO-A 03/344011, WO-A 03/24959, WO-A 03/04457, WO-A 03/04450, WO-A 01/72706, WO-A 98/04543, U.S. Pat. No. 5,298,627, WO 89/07598 or in Tetrahedron Letters (1992), 33 (17), 2283-4.

The following examples explain the invention.

### EXAMPLES

The following examples relate to the following synthesis scheme given by way of example:

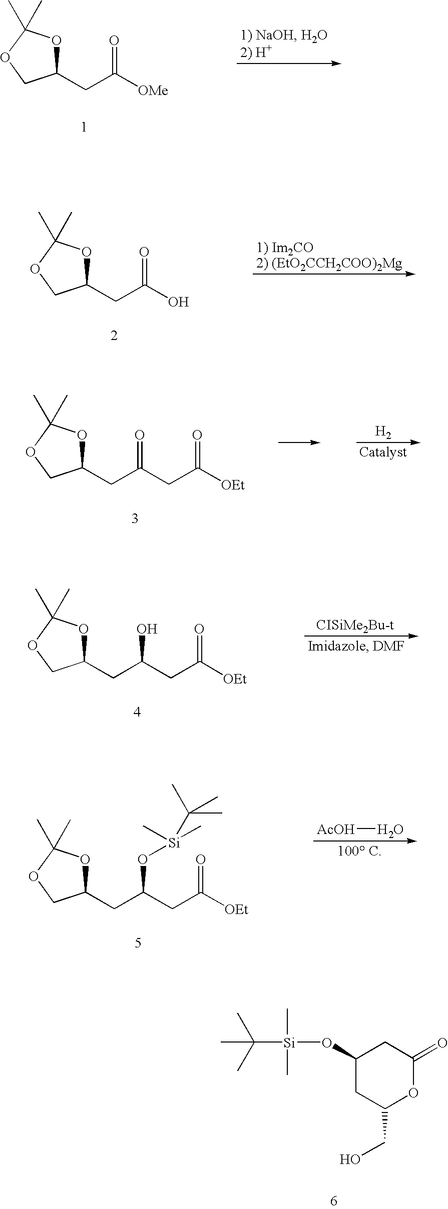

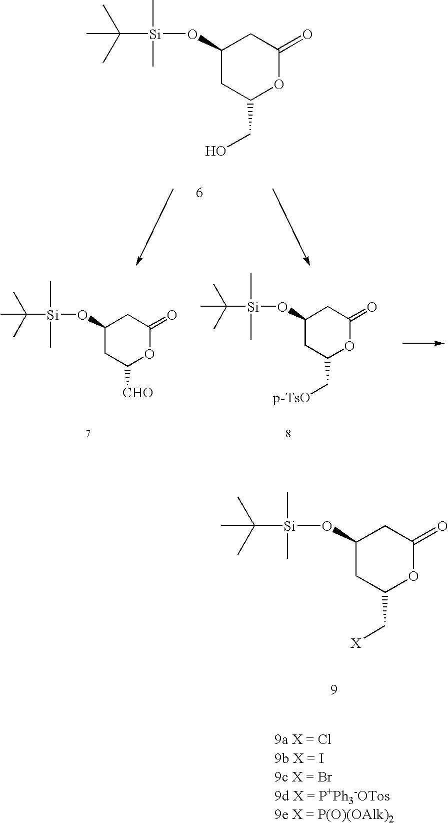

### Example 1

### Methyl (4S)-(2,2-dimethyl-1,3-dioxolan-4-yl)acetate (compound 1)

This compound is commercially available, for example from Aldrich or it can be prepared in a simple manner starting from methyl(S)-malate, one of the ester groups being selectively reduced according to Chem. Letters 1984, 1389-1392, or Tetrahedron 1992, 48, 4067-4086.

0.28 g (0.0074 mol) of NaBH₄ was added in one portion to a solution of 113.4 g (0.70 mol) of dimethyl(S)-malate in 300 ml of absolute THF. 68 ml (54.5 g, 0.72 mol) of the BH₃.Me₂S complex were then slowly added at room temperature with stirring. During the addition, gaseous products were evolved. After the end of the addition, the reaction mixture was kept at room temperature for 3 hours. Thereafter, 285 ml of methanol were added, and the solution obtained was left to stand overnight at room temperature. The volatile constituents were evaporated off, and the viscous residue was dried for 6 hours under reduced pressure. The residue was mixed with 300 ml of acetone, 96.3 ml (81.6 g, 0.78 mol) of Me₂C(OMe)₂ and 4 g (0.021 mol) of p-TsOH.H₂O. The reaction was stirred overnight at room temperature. Neutralization was then effected with 4 g of sodium carbonate. The reaction mixture was stirred for 1 further hour, filtered and evaporated. The residue was distilled under reduced pressure (74° C./6 mbar), and 90.6 g (74.4%) of compound 1 were obtained.

### Example 2

### (4S)-(2,2-dimethyl-1,3-dioxolan-4-yl)acetic acid (compound 2)

50 g (0.287 mol) of the compound of example 1 were added with stirring to a 2 molar aqueous sodium hydroxide solution (287 ml, 0.574 mol) cooled with ice. The ice bath was removed, and the mixture was stirred for 2 hours. The mixture was extracted with dichloromethane (3×50 ml), and the organic extracts were separated off. The aqueous layer was mixed with 100 ml of diethyl ether and cooled with ice. 300 ml of 2 normal aqueous sodium hydrogen sulphate solution were added to the mixture. The mixture was stirred vigorously for 15 minutes. The organic phase was separated off, and the aqueous phase was extracted with ethyl acetate (2×100 ml). The combined organic phases were dried with sodium sulphate and evaporated. The residue was dried under reduced pressure, and 36 g (78.3%) of a liquid product were obtained. The purity of the end product, determined by means of an NMR spectrum, was 95%. The product could be used without further purification.

¹H NMR (CDCl₃), δ in ppm: 1.37 (3H, s), 1.43 (3H, s), 2.58 (1H, dd, J=16.2 and 6.7 Hz), 2.75 (1H, dd, J=16.2 and 6.7 Hz), 3.68 (1H, dd, J=8.5 and 6.1 Hz), 4.17 (dd, J=8.5 and 6.0 Hz), 4.45-4.53 (1H, m), 11 (1H, br. s).

¹³C NMR (CDCl₃), δ in ppm: 25.8 (CH₃), 27.2, (CH₃), 39.2 (CH₂), 69.4 (CH₂), 72.1 (CH), 109.9 (C), 176.7 (COO).

### Example 3

### Ethyl (4S)-(2,2-dimethyl-1,3-dioxolan-4-yl)-3-oxohexanoate (compound 3)

A mixture of 49.0 g (0.371 mol) of monoethyl malonate and 6.0 g (0.248 g-atom) of magnesium were refluxed in 200 ml of absolute THF for four hours with stirring, with the result that a first solution was obtained. Simultaneously with this, 28.8 g (0.177 mol) of solid N,N′-carbonylbisimidazole were added in the course of 5 to 10 minutes to a solution of 25.8 g (0.161 mol) of the compound of example 2 in 100 ml of absolute THF, gas evolution occurring. Stirring was then effected for 2 hours at room temperature. The cooled first solution was then added to this solution. The remaining magnesium was washed with 50 ml of absolute THF, and the wash solution was then added to the reaction mixture. The reaction mixture was stirred overnight at room temperature. The reaction mixture was evaporated, and the residue was dissolved in 200 ml of ethyl acetate and was acidified with 430 ml of a 2 normal aqueous sodium hydrogen sulphate solution with vigorous stirring. The organic phase was separated off, washed in succession with 2 normal aqueous sodium hydrogen sulphate solution (2×200 ml) and saturated aqueous sodium bicarbonate solution (3×200 ml), dried over sodium sulphate and evaporated. The residue was distilled under reduced pressure (90 to 93° C., 0.07 mbar), and 26.9 g (72.4%) of compound 3 were obtained. The NMR spectra in CDCl₃ showed that the product contained about 10% of the enol form. The following NMR spectrum relates exclusively to the keto form.

¹H NMR (CDCl₃), δ in ppm: 1.28 (3H, t, J=7.1 Hz), 1.35 (1H, s), 1.40 (1H, s), 2.75 (1H, dd, J=17.1 and 6.8 Hz), 2.99 (1H, dd, J=17.1 and 6.1 Hz), 3.49 (2H, s), 3.57 (1H, dd, J=8.4 and 6.6 Hz), 4.15-4.24 (3H, m), 4.42-4.52 (1H, m).

¹³C NMR (CDCl₃), δ in ppm: 14.3 (CH₃), 25.7 (CH₃), 27.1 (CH₃), 47.4 (CH₂), 49.9 (CH₂), 61.7 (CH₂), 69.5 (CH₂), 71.7 (CH), 109.2 (C), 167.1 (COO), 201 (C═O).

### Example 4

### Ethyl (3R,4S)-(2,2-dimethyl-1,3-dioxolan-4-yl)-3-hydroxyhexanoate (compound 4)

a) Preparation of the Catalyst.

A mixture of 200 mg (0.295 mmol) of (R)-TolBINAP, 73.6 mg (0.147 mmol) of [Ru(C₆H₆)Cl₂]₂ and 2 ml of DMF were stirred at 100° C. for 15 minutes under argon. The volatile constituents were evaporated off, and the residue was dried under reduced pressure for 1 hour at 50° C. This process for the preparation of BINAP catalysts is based on the publication in Tetrahedron Lett. 1991, 32, 4163. The residue was dissolved in 3 ml of dichloromethane, and 0.2 ml of triethylamine was added. After 1 hour at room temperature, the volatile constituents were evaporated off, and the residue was dried under reduced pressure. The solid product was used as a catalyst for the following hydrogenation without further purification and characterisation.

b) Hydrogenation of the Ketone of Example 3

A mixture of 0.58 g (2.5 mmol) of the compound of example 3 and 4.3 mg (about 0.005 mmol) of the precatalyst prepared in step a), in 10 ml of absolute oxygen-free methanol, was hydrogenated under initially 50 bar hydrogen pressure at room temperature with stirring under anaerobic conditions. After 150 minutes the hydrogen adsorption was complete. The autoclave was opened and the mixture was evaporated and dried under reduced pressure. The conversion and the yield were quantitative. According to the NMR spectra, the diastereomeric purity of the product was greater than 99%. The diastereomeric purity was determined on the basis of the NMR spectra by analogy with the corresponding methyl esters according to Chem. Ber. 1998, 2035-2044.

¹H NMR (CDCl₃), δ in ppm: 1.27 (3H, t, J=7.1 Hz), 1.36 (3H, s), 1.42 (3H, s), 1.72-1.83 (2H, m), 2.45-2.59 (2H, m), 3.53-3.61 (2H, m), 4.08-4.35 (5H, m).

¹³C NMR (CDCl₃), δ in ppm: 14.4 (CH₃), 25.9 (CH₃), 27.1 (CH₃), 39.9 (CH₂), 41.8 (CH₂), 60.8 (CH₂), 67.0 (CH), 69.7 (CH₂), 74.6 (CH), 109.4 (C), 172.3 (COO).

### Example 5

### (3R,4S)-(2,2-dimethyl-1,3-dioxolan-4-yl)-3-(tert-butyldimethylsilyloxy)hexanoate (compound 5)

The title compound was obtained in a yield of 88% from the compound of example 4 according to the method in J. Org. Chem. 1984, 49, 3994-4003.

### Example 6

### (4R,6S)-4-(tert-butydimethylsilyloxy)-6-hydroxymethyltetrahydropyran-2-one (compound 6)

The title compound was obtained in a yield of 60% from the compound of example 5 according to the process in J. Org. Chem. 1984, 49, 3994-4003.

### Example 7

### (4R,6S)-4-(tert-butyldimethylsilyloxy)-6-(p-toluenesulphonyloxymethyl)tetrahydropyran-2-one (compound 8)

The title compound was obtained in a yield of 91% from the compound of example 6 according to the process in J. Org. Chem. 1984, 49, 3994-4003.

1. A process for the preparation of a statin, comprising the following steps:
a) preparing a compound of the formula II 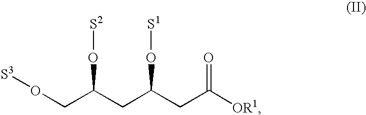
in which
S¹ denotes a hydrogen atom or a hydroxylprotective group,
S² and S³, independently of one another, denote hydroxylprotective groups and
R¹ represents a hydrogen atom or a carboxylprotective group,
by stereoselective hydrogenation of a compound of the formula III 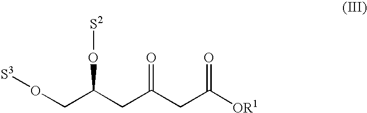
to give a compound of the formula II-a 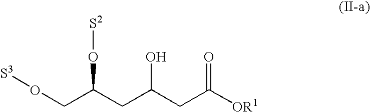
and optionally introducing a hydroxylprotective group; and
b) lactonizing the compound of the formula II to give a compound of the formula I-a

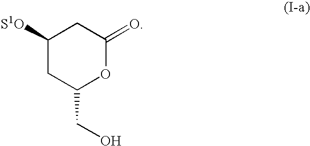

2. The process according to claim 1, comprising the further step of
c) converting the compound of the formula I-a 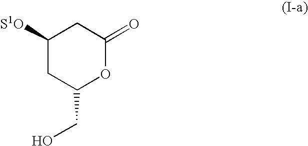
into a compound of the formula I 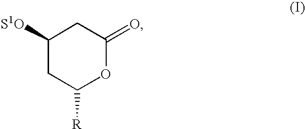
wherein the radical
S¹ is as defined in claim 1,
R denotes —CH₂R², —CHO, —CH═P(R³)₃, —CH₂—P⁺(R³)₃M⁻, 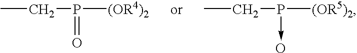
R² denotes a halogen atom, —C≡N, —CH₂NH₂, —SO₂—R⁶ or a leaving group,
R³, R⁴ and R⁵ complete a Wittig radical or a Horner-Wittig radical,
R⁶ denotes a hydrogen atom or a C₁₋₃-alkyl or a C₅₋₁₀-aryl radical, which are optionally substituted by one or more radicals which, independently of one another, are selected from halogen atoms, heterocycles which contain 0 to 10 carbon atoms and 1 to 10 heteroatoms selected from sulphur, nitrogen and oxygen atoms, and functional groups and
M⁻ represents an opposite ion.

3. The process according to claim 1, comprising the step of:
preparing a compound of the formula III 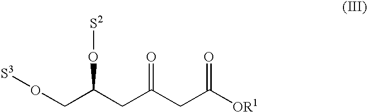
by chain extension of a compound of the formula IV

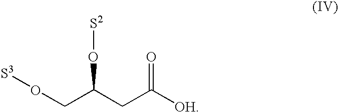

4. The process according to claim 1, wherein the compound of the formula I is converted into the statin by one of the following processes and then optionally opening the lactone ring and optionally removing the protective groups:
a) reacting a compound of the formula (I) 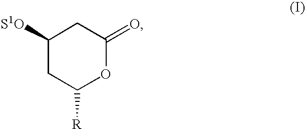
in which the radical R represents a CHO group and the radical S¹ is as defined in claim 1, with a compound of the formula 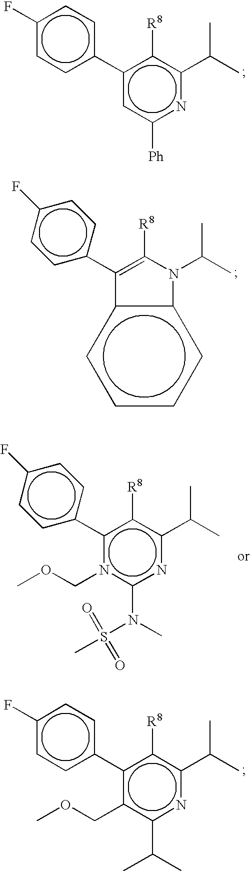
in which
R⁸ denotes —CH═P(R³)₃, —CH₂—P⁺(R³)₃M⁻, 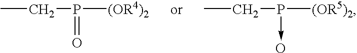
where R³, R⁴, R⁵ and M are as defined in claim 1,
b) reacting a compound of the formula I 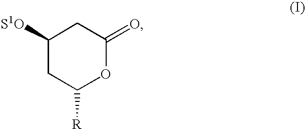
in which
the radical R denotes —CH═P(R³)₃, —CH₂—P⁺(R³)₃M⁻, 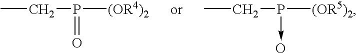
with a compound of the formula 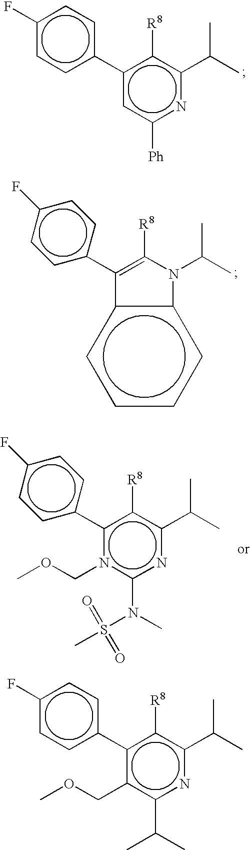
in which
R⁸ denotes —CHO,
where R³, R⁴, R⁵ and M are as defined in claim 1,
c) reacting a compound of the formula I 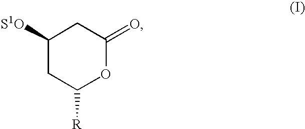
in which
the radical R is a group —CH₂—C≡N, hydrogenating the compound of the formula I in which the radical R is a group —CH₂—C≡N, to give a compound of the formula I in which the radical R is a group —CH₂—CH₂NH₂, and reacting the compound of the formula I in which the radical R is a group —CH₂—CH₂NH₂ with a compound of the formula V 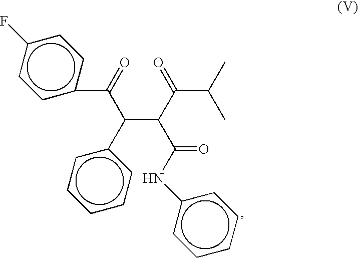
d) hydrogenating a compound of the formula I 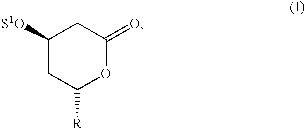
in which
the radical R is a group —CH₂—C≡N, to give a compound of the formula I in which the radical R is a group —CH₂—CH₂NH₂,
and reacting the compound of the formula I in which the radical R is a group —CH₂—CH₂NH₂ with a compound of the formula V 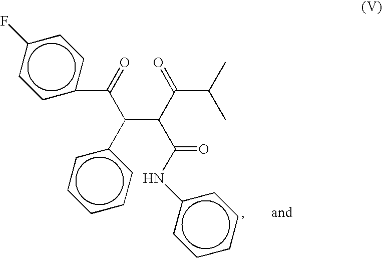
and
e) reacting a compound of the formula (I) 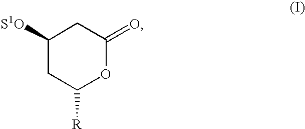
in which
the radical R is a group —CH₂—CH₂NH₂, with a compound of the formula V

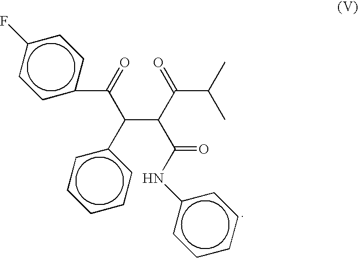

5. The process according claim 1, characterized in that a compound of the formula 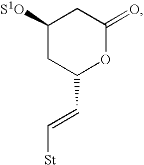
in which S¹ is as defined in claim 1 and St represents the radical of the statin, is converted into a compound of the formula 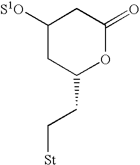
by catalytic hydrogenation, and optionally the protective group S¹ is removed and optionally the lactone ring is opened.

6. The process according to claim 1, wherein the hydroxylprotective group S¹ is selected from a trimethylsilyl, triisopropylsilyl, trimethylsilylethyl, tert-butyldimethylsilyl, tert-butylmethylsilyl, di-tert-butylmethylsilyl, tert-butyldiphenylsilyl, triphenylsilyl, diphenylmethylsilyl, tris(trimethylsilyl) and para-tosyl protective group.

7. The process according to claim 1, wherein the protective groups S² and S³ are bridged.

8. The process according to claim 7, wherein the protective groups S² and S³ together represent an isopropylidene protective group.

9. The process according to claim 2, wherein the radical R represents a radical CH₂R² and R² represents a leaving group, the leaving group being selected from a halogen atom and a radical —OSO₂—C₁-C₆-alkyl, and —OSO₂—C₅-C₁₀-aryl.

10. The process according to claim 1, wherein the radical R¹ denotes a hydrogen atom, a C₁₋₃-alkyl, or a C₄₋₁₀-aryl radical, each of which may be optionally substituted by one or more radicals, which, independently of one another, are selected from halogen atoms, heterocycles which have 0 to 10 carbon atoms and 1 to 10 heteroatoms selected from sulphur, nitrogen and oxygen atoms, and functional groups.

11. The process according to claim 1, wherein
R³ denotes a C₅- to C₁₀-aryl radical which is optionally substituted by one or two C₁-C₄-alkyl radicals and/or halogen atoms, a C₁-C₄-alkyl radical or a C₅-C₁₀-cycloalkyl radical,
R⁴ denotes a C₁-C₄-alkyl radical, and
R⁵ denotes a C₁-C₆-alkyl or C₅-C₁₀-aryl radical.

12. The process according to claim 1, wherein the statin is fluvastatin, rosuvastatin, cerivastatin, glenvastatin or atorvastatin.

13. A compound of the formula I 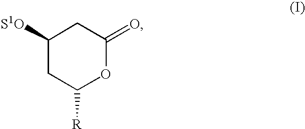
in which
S¹ and R are as defined in claim 2, with the proviso that the radical S¹ does not represent a tert-butyldimethylsilyl group if the radical R represents a CHO, —CH₂—OTos, —CH₂Cl or —CH₂I group.

14. A compound according to claim 13, in which the radical S¹ represents a tert-butyldimethylsilyl group and the radical R represents a —CH₂R², —CH═P(R³)₃, —CH₂—P⁺(R³)₃M⁻, 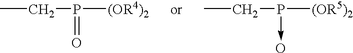
group, wherein R² represents a bromine atom, a —C≡N, a —CH₂NH₂ group or a radical —SO₂—R⁶, and R³, R⁴, R⁵, R⁶ and M are as defined in claim 2.

15. The process for the preparation of a compound of a formula (I-a) 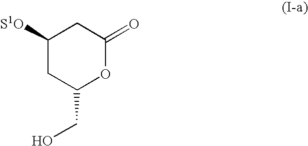
in which the radical S¹ is as defined in claim 1, characterized in that a compound of the formula II 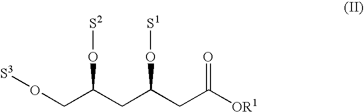
in which
S¹, S², S³ and R¹ are as defined in claim 1, is converted into the compound of the formula I-a by lactonization.
